(19) United States
(12) Patent Application Publication
(10) Pub. No.: US 2005/0287222 A1
(43) Pub. Date: Dec. 29, 2005

(54) Method for producing water containing extracted ingredients from plant, animal, or mineral matter

(76) Inventor: Henry Aoki, Acton, MA (US)

Correspondence Address: Lipsitz & McAllister, LLC, 755 MAIN STREET, MONROE, CT, 06468, US

(21) Appl. No.: 10/875,390

(22) Filed: Jun. 24, 2004

(51) Int. Cl. A61K 35/14; A61K 35/78

(52) U.S. Cl. 424/529; 424/725

(57) ABSTRACT

Water is produced containing extracted ingredients from plant, animal and/or mineral matter. Raw material is placed into contact with an absorber material, which holds the raw material after drying. Atomized heated water is applied to the absorber material in a depressurized state to extract effective ingredients from the raw material. The atomized particles containing the extracted ingredients are circulated with air and delivered to a cooled condenser. Water containing the effective ingredients is collected in a reservoir tank after being condensed by the condenser. Atomized fine particles that are not condensed are recirculated.

### BACKGROUND OF THE INVENTION

1. Field of the Invention

The present invention relates to water containing extracted ingredients holding effective ingredients extracted from plant matter, animal matter, and mineral matter, and to a method of production thereof.

2. Description of the Related Art

Among others, Japanese patent No. 323387 and U.S. Pat. No. 5,558,006 disclose technologies for the holding in solution of effective ingredients extracted from plant matter, animal matter, and mineral matter.

These conventional extraction apparatuses comprise an atomized fine particle generating tank provided with a heater heating stored water to a predetermined temperature and a means for atomization of the water; an extraction device holding a raw material layer and connected to the atomized fine particle generating tank in order to make the raw material's effective ingredients adhere to the atomized fine particles when the atomized fine particles pass therethrough; a condenser connected to the extraction device and condensing the atomized fine particles having passed through the raw material layer; a reservoir tank for installation of the water condensed in the condenser; a blower disposed on the path between the reservoir tank and the atomized fine particle generating tank in order to depressurize the material layer in the extraction device; and a cooling means for cooling of the extraction device and the reservoir tank.

However, in situations where the raw material is a liquid, extraction of ingredients therefrom is not possible using these conventional technologies. In other words, liquids or fluidity substances such as blood, animal milk, starch syrup, wood acid, resin, and sap, etc. cannot be subjected to extraction using these technologies, and hence, effective ingredients cannot be extracted therefrom.

In addition, when solid raw material is subjected to extraction without any pre-processing, the area of contact with the atomized fine particles is small and extraction efficiency is impaired as a result.

### SUMMARY OF THE INVENTION

The present invention relates to a producing method for water containing extracted ingredients from plant, animal, and mineral matters comprises the steps of;
(a) making a liquid or fluidity raw material come into contact with an absorber material and drying the absorber material to obtain a holding body of the raw material;
(b) generating atomized fine particles of water heated using a heater heating stored water to a predetermined temperature and an atomized fine particle generating tank providing a means for atomization of the water;
(c) depressurizing a raw material layer comprising holding body according to the step (a) charged in an extraction device and sucking to and exuding out the surface of the atomized fine particles the effective ingredients in the raw materials;
(d) making the effective ingredients deposited on the raw material surface through suction and exudation hold onto the atomized fine particles by circulating the atomized fine particles together with a flow of air in a cyclinc fashion through the various component devices and passing thorugh the depressurized raw material layer;
(e) delivering the atomized fine particles holding the effective ingredients to a cooled condenser and condensing the particles;
(f) instilling into a reservoir tank the water containing the effective ingredients of the various raw materials after being condensed by the condenser, and obtaining the final product;
(g) re-circulating to the atomized fine particle generating tank the atomized fine particles not ocndensed in the condenser.

Further, the absorber material from the above-described producing method may comprise polyvinylidene fluoride, glass fiber, or cellulose.

In addition, a liquid or fluidity substance comprising crushed or pulverized pieces of plant matter, animal matter, or mineral matter together with the required amount of water may be used as the raw material in any of the above-described producing methods.

Furthermore, blood, animal milk, starch syrup, wood acid, resin, sap, yogurt, cheese, miso soybean paste, soy sauce, fish sauce, soya milk, crystal-powder aqueous solutions, tourmaline-powder aqueous solutions, or pearl-powder aqueous solutions may be used as the liquid or fluidity raw material in any of the above-described producing methods.

Further, in any of the above-described producing methods, the temperature of the water in the atomized fine particle generating tank is approximately 80° C. or lower, and the temperature of the raw material layer and the atomized fine particles in the extraction device may be between approximately 60° C. and 70° C.

In addition, the present invention provides water containing extracted ingredients principally including ingredients extracted from plant, animal, and mineral matters and produced according to the following processes of;
(a) making a liquid or fluidity raw material come into contact with an absorber material and drying the absorber material to obtain a holding body of the raw material;
(b) generating atomized fine particles of water heated using a heater heating stored water to a predetermined temperature and an atomized fine particle generating tank providing a means for atomization of the water;
(c) depressurizing a raw material layer comprising holding body according to the process (a) charged in an extraction device and sucking to and exuding out the surface of said atomized fine particles the effective ingredients in the raw materials;
(d) making the effective ingredients deposited on the raw material surface through suction and exudation hold onto the atomized fine particles by circulating the atomized fine particles together with a flow of air in a cyclinc fashion through the various component devices and passing thorugh the depressurized raw material layer;
(e) delivering the atomized fine particles holding the effective ingredients to a cooled condenser and condensing the particles;
(f) instillating into a reservoir tank the water containing the effective ingredients of the various raw materials after being condensed by the condenser, and obtaining the final product;
(g) re-circulating to the atomized fine particle generating tank the atomized fine particles not condensed in the condenser.

Further, the absorber material for the above-described water containing extracted ingredients may comprise polyvinylidene fluoride, glass fiber, or cellulose.

In addition, a liquid or flowing body comprising crushed or pulverized pieces of plant matter, animal matter, or mineral matter together with the required amount of water may be used as the raw material for any of the above-described water containing extracted ingredients.

Furthermore, blood, animal milk, starch syrup, wood acid, resin, sap, yogurt, cheese, miso soybean paste, soy sauce, soya milk, fish sauce, crystal-powder aqueous solutions, tourmaline-powder aqueous solutions, or pearl-powder aqueous solutions may be used as the liquid or fluidity raw material for any of the above-described water containing extracted ingredients.

Further, for any of the above-described producing methods, the temperature of the water in the atomized fine particle generating tank is approximately 80° C. or lower, and the temperature of the raw material layer and the atomized fine particles in the extraction device may be between approximately 60° C. and 70° C.

### BRIEF DESCRIPTION OF THE DRAWINGS

FIG. 1 is a block diagram showing the construction of a producing apparatus and producing method.

FIG. 2 is an external perspective view of the producing apparatus.

FIG. 3 is an external perspective view of the interior of the cold storage chamber of the producing apparatus.

FIG. 4 is an external perspective view of the external cylinders of the extraction device.

FIG. 5 is an external perspective view showing the construction of the internal cylinder of the extraction device.

### DESCRIPTION OF THE PREFERRED EMBODIMENTS

A liquid or fluidity substance comprising crushed or pulverized pieces of plant matter, animal matter, or mineral matter together with the required amount of water is used as the raw material for ingredient extraction. Certain substances liquefy naturally upon crushing or pulverization, and in such a case, the absorber material is immersed in the liquefied substance. Further, in the case of mineral substances that do not liquefy naturally upon crushing or pulverization, water is added thereto and the absorber material is immersed in the slurry obtained. Whereas blood, animal milk, starch syrup, wood acid, resin, sap, yogurt, cheese, miso soybean paste, soy sauce, fish sauce, soya milk, crystal-powder aqueous solutions, tourmaline-powder aqueous solutions, and pearl-powder aqueous solutions, etc. are identified as raw materials, this list is not restrictive, and any crushed or pulverized substance mixed with water can be used. Accordingly, for example, internal organs and a wide range of other parts of fish and other animals can be used as the raw material.

An absorbent material with no nutritional value is used as the absorber material. Further, it is preferable for the absorber material to have a plurality of pores in accordance with the fact that contact efficiency between the atomized fine particles explained hereinafter and the raw material adhering to the absorber material increases when the atomized fine particles pass through pores therein.

Suitable materials include hydrophilic membrane filters such as modified polyvinylidene fluoride membranes such as the Durapore® filter commercially available from Millipore Corporation, glass fiber membranes, cotton, nylon, cellulose, or paper material such as that used in tea bags. The form of the material is not particularly limited, and can include sheets add discs. The particular identity of the material chosen for a given application will depend in part upon the nature of the solvent used in a subsequent process, such as an analysis process used to identify the ingredients of the final product.

Next, the absorber material immersed in the liquid or fluidity raw material and to which the raw material has adhered is dried in order to obtain a holding body of the raw material.

Although freeze-drying, heat drying, and air-flow drying are acceptable, freeze-drying is preferable. In this drying process, the raw material held by the absorber material is also dried. Subsequently, in the production device explained hereinafter, the extraction device's raw material layer is formed using the above-described holding body of the raw material and extraction is carried out. More specifically, the production device comprises, an atomized fine particle generating tank provided with a heater heating stored water to a predetermined temperature and a means for atomization of said water; an extraction device holding a raw material layer and connected to the atomized fine particle generating tank in order to make the raw material's effective ingredients adhere to the atomized fine particles when the atomized fine particles pass therethrough; a condenser connected to the extraction device and condensing the atomized fine particles having passed through the raw material layer; a reservoir tank for installation of the water condensed in the condenser; a blower disposed on the path between the reservoir tank and the atomized fine particle generating tank in order to depressurize the material layer in the extraction device; and a cooling means for cooling of the extraction device and the reservoir tank. All components such as tanks, devices, and means are connected by pipe capable of circulating air through. An ultrasonic vibrator is used as the atomization means. Although the heater's heating temperature varies with respect to the raw material subjected to extraction, a temperature between a range of 60° C. and 70° C. is generally preferable, and temperatures in excess of 80° C. are not required.

The absorber material holding the dried raw material to be subjected to extraction is placed inside the extraction device. The blower operates and the space housing the raw material inside the extraction device is depressurized; then, the minute atomized particles from the atomized fine particle generating tank reach the extraction device, freely pass through the pores in the absorber material holding the raw material, capture the ingredients having exuded to the surface thereof, and move to the condenser.

The condenser cools the atomized fine particles using a suitable means; accordingly, condensed atomized fine particles are delivered to the reservoir tank.

This condensate is water containing extracted ingredients realized through extraction and capture of raw material ingredients. The present invention is described in more detail hereinafter with reference to the preferred embodiments thereof.

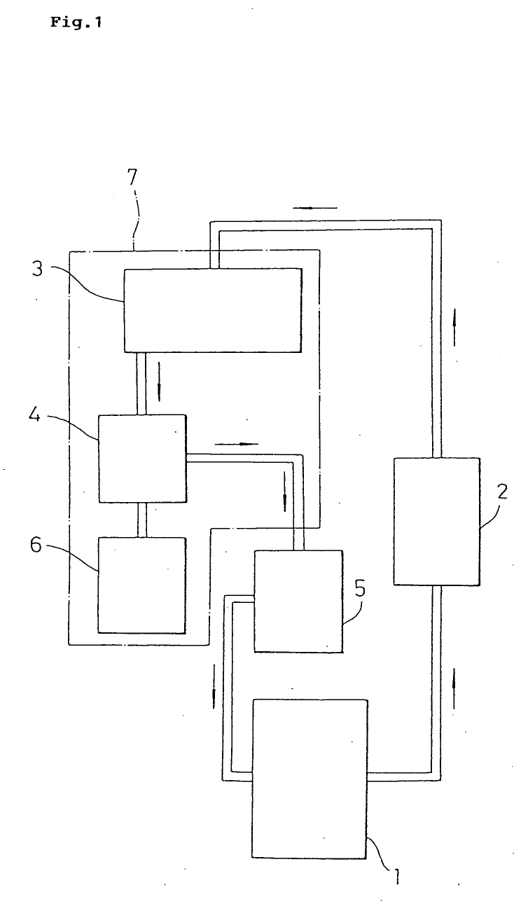

FIG. 1 is a block diagram showing the construction of the first embodiment of a producing apparatus in which 1 is an atomized fine particle generating tank; 2 is an extraction device extracting effective ingredients from blood and other raw materials held by an absorber material using atomized fine particles delivered from the atomized fine particle generating tank 1; 3 is a condenser condensing the atomized fine particles holding the effective ingredients from the raw materials transferred from the extraction device 2; 4 is a reservoir tank for instillation of the water containing the effective ingredients of the raw materials condensed at the condenser 3; 5 is a blower disposed between the reservoir tank 4 and the atomized fine particle generating tank 1.

In addition, 6 is a secondary reservoir tank connecting with the reservoir tank 4, and 7 is a cooling means for cooling of the condenser 3, reservoir tank 4, and secondary reservoir tank 6. As shown in the figure, the atomized fine particle generating tank 1, the extraction device 2, and the other devices are inter-connected by connecting pipes thus forming a circulatory route centered around the atomized fine particle generating tank 1, and atomized fine particles pass in a cyclic fashion together with the flow of air around this circulatory route as a result of the action of the blower 5.

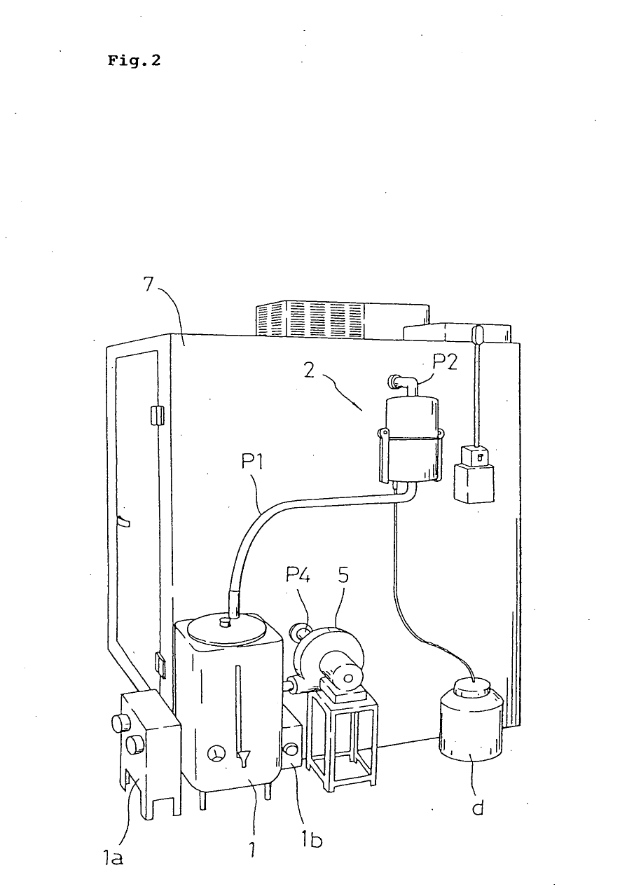

FIG. 2 is an external perspective view of a producing apparatus having the construction explained above. In this figure, 1 is an atomized fine particle generating tank comprising a stainless-steel water tank of 35 cm in width, 35 cm in length, and 60 cm in height, in which between 30 and 40 liters of water is stored during operation. 1a is ultrasonic generator having eight pairs of oscillators disposed at the bottom of the water tank 1, each of which is capable of atomizing approximately 0.5 liters of water per hour. 1b is a heater for setting the water in the water tank 1 to a predetermined temperature.

Furthermore, 2 is an extraction device described hereinafter disposed on the side wall of a cold storage chamber 7 constituting the cooling means and connected to the atomized fine particle generating tank 1 by a flexible plastic pipe P1 of 38 mm in diameter and approximately 1.3 m in length. Note that d is a discharged water tank for receiving moisture discharged from the extraction device 2. P2 is a pipe connecting the extraction device 2 to the condenser 3 described hereinafter and constituted by a metal pipe of 40 mm in diameter.

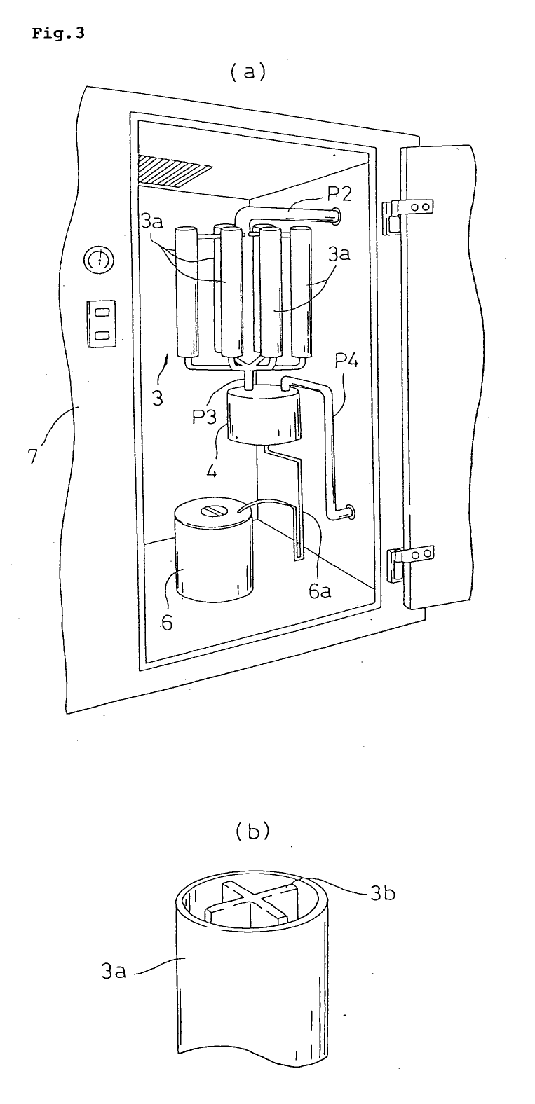

FIG. 3 (a) is an external perspective view of the interior of the cold storage chamber 7 in which 3 is a condenser constituted by a multiplicity (six in this embodiment) of condensing tubes 3a, and as explained above, connected to the extraction device 2 disposed outside the cold storage chamber 7 by the communication pipe P2. In this embodiment, each condensing tube 3a is a metal pipe of 85 mm in diameter and approximately 550 mm in length, and as shown in FIG. 3 (b), a cooling plate 3b is provided inside each condensing tube 3a. The top of each condensing tube 3a is connected to the communication pipe P2 by a branch pipe, and similarly, the bottom thereof is connected to a communication pipe P3 by a branch pipe.

4 is a reservoir tank connected to the condenser 3 by a communication pipe P3 of 40 mm in diameter and provided for the instillation of water liquefied from the atomized fine particles at the condenser 3. The top of the reservoir tank 4 and the blower 5 disposed outside the cold storage chamber 7 are connected by a communication pipe P4 of 40 mm in diameter. 6 is a secondary reservoir tank connected to the reservoir tank 4 by a drain pipe 6a. The cooling device is provided on the ceiling of the cold storage chamber 7; however, a window-type air conditioner may be installed on the side wall thereof in order to increase cooling performance.

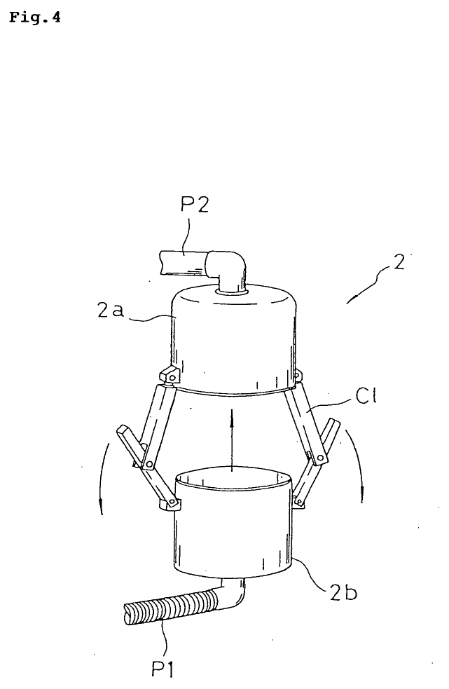

FIG. 4 is an external perspective view of the external cylinders constituting components of the extraction device 2 and comprising a primary external cylinder 2a and a secondary external cylinder 2b, both of which are supported with capability for free joining and separation through the action of a clamp C1, and each of which is a stainless steel member with a cylindrical shape of approximately 200 mm in diameter and approximately 150 mm in depth. Note that a temperature sensor for sensing of the temperature during extraction operation is attached to the lower secondary external cylinder 2b.

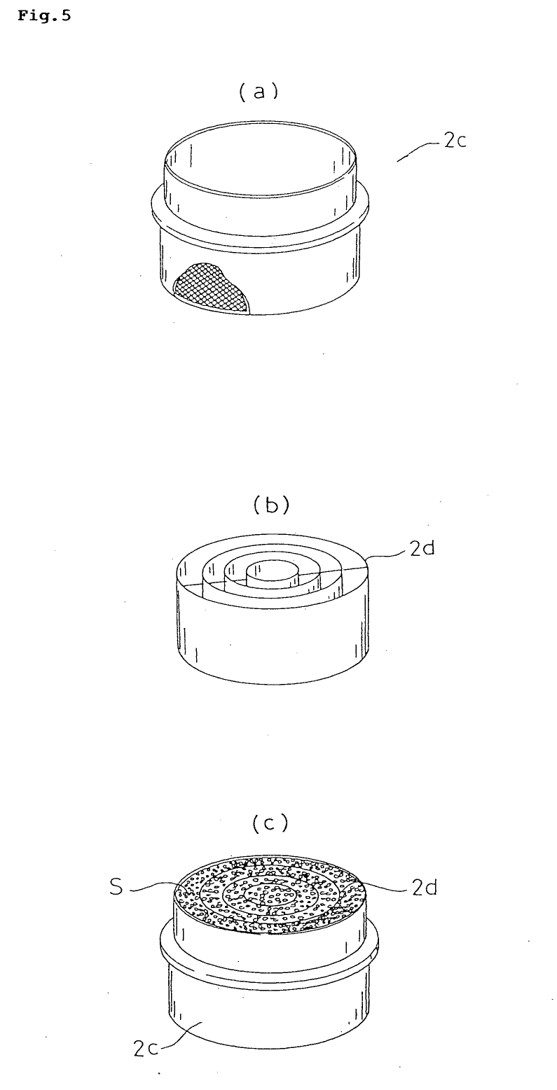

FIG. 5 is a drawing explaining the inner cylinder constituting a structural element of the extraction device 2, and FIG. 5 (a) shows a perspective view of the inner cylinder 2c. The inner cylinder 2c is formed so as to have dimensions allowing press fitting thereof into the outer cylinders, and a net for holding the holding body of the raw material (an absorber material to which dried raw material is adhered) is provided at the bottom thereof. FIG. 5 (b) shows guide plates 2d for insertion into the inner cylinder 2c, and as shown in FIG. 5 (c), the holding body of the raw material, that is a glass fiber membrane constituted by the dried absorber material to which a desired raw materials such as blood, animal milk, starch syrup, wood acid, resin, sap, yogurt, cheese, miso soybean paste, soy sauce, soya milk, fish sauce, crystal-powder aqueous solutions, tourmaline-powder aqueous solutions, or pearl-powder aqueous solutions has adhered, is partitioned and stored thereby within the inner cylinder 2c.

The presence of the guide plates 2d has the effect of facilitating smoother passage of the atomized fine particles as described hereinafter. Note that these guide plates 2d can also be formed with a spiral shape.

As explained above, the extraction device 2 comprises a pair of external cylinders and an inner cylinder fit therein.

Hereinafter, the operation of the producing apparatus and the preferred embodiments of the production method thereof will be described in accordance with the construction explained above.

Chicken blood and cow's milk shall be used as the raw material in this embodiment. A 500 ml of this blood or milk was absorbed into an absorber material fabricated from glass fiber, and the absorber material was then freeze-dried. Although the blood or milk adheres to individual glass fibers in the dried solid form thereof, a plurality of pores exists between the glass fibers, allowing the atomized fine particles to pass freely therebetween. Furthermore, the absorber material is cut into small pieces in advance.

First of all, the inner cylinder 2c as shown in FIG. 5 (a) is charged with the small cut pieces of the above-described absorber material. At this time, the weight of the absorber material is approximately 800 grams including the weight of the dried blood or milk. Upon charging, the guide plates 2d as shown in FIG. 5 (b) are disposed inside the inner cylinder 2c. When charging has been completed, the absorber material can be covered with a net to hold it securely within the inner cylinder 2c.

Next, the inner cylinder 2c is press fit into the extraction device 2. While this is occurring, between 30 and 50 liters of water is stored in the atomized fine particle generating tank 1 as shown in FIG. 2. Note that the atomized fine particle generating tank 1 is constructed in such a way that the volume of water mentioned above can be automatically maintained.

When the water disposed in the atomized fine particle generating tank 1 and the above-described absorber material holding the raw material and disposed inside the extraction device 2 have been setup as required, the temperature of the water inside water tank 1 is raised using the heater 1b of the atomized fine particle generating tank 1. Experience has shown that a temperature setting of 85° C. is most suitable. This temperature of 85° C. is, as described hereinafter, the most suitable for maintaining a temperature of between 60° C. and 70° C. within the extraction device 2.

When the temperature of the water inside the water tank 1 reaches the set temperature of 85° C., the switch for the ultrasonic generator 1a turns on, as does the switch for the blower 5. As a result of the operation of the blower 5, the air flow circulates through a circulatory route formed by the atomized fine particle generating tank 1, the extraction device 2, the condenser 3, the reservoir tank 4, the blower 5, and the communication pipes connecting these components.

Together with the air flow, therefore, the atomized fine particles of water from the atomized fine particle generating tank 1 pass through the plastic pipe P1 and arrive at the extraction device 2. Note that as explained above, a temperature in the range of 60° C. to 70° C. is preferable for the atomized fine particles in the extraction device 2. For this reason, the temperature inside the extraction device 2 is constantly detected by a temperature sensor attached thereto, and in order to achieve the preferable temperature, the temperature in the atomized fine particle generating tank 1 is controlled based on the detected results from the sensor.

Although the air flow circulates through the various devices as a result of the action of the blower 5 as explained above, absorber materials absorbed by raw materials are charged into the extraction device 2, and the air flow having passed through the plastic pipe P1 thus experiences resistance and the flow thereof is obstructed. In contrast, no obstruction to the passage of the air flow exists in the communication pipe P2 or any downstream components of the circulatory route. Consequently, the air inside the extraction device 2 becomes depressurized.

When the air inside the extraction device 2 becomes depressurized, known and unknown ingredients contained within the dried blood or milk constituting the raw materials exude to the surface thereof. The passing atomized fine particles capture the various ingredients exuded to the surface of the blood or the milk. As explained above, the temperature within the extraction device 2—or more specifically, the temperature within the inner cylinder 2c—is maintained at approximately 65° C., and therefore, the ingredients contained in the blood are extracted into the atomized fine particles without being destroyed by heat.

The atomized fine particles containing the effective ingredients from the blood or the milk pass together with the air flow through the connecting pipe P2 and reach the condensing tubes 3a of the condenser 3. The condensing tubes 3a and the cooling plates 3b contained therein are disposed within the cold storage chamber 7 to be cooled, and therefore, the atomized fine particles coming into contact therewith are liquefied and change to water containing extrcted ingredients from the blood or the milk. The water containing extracted ingredients from the blood or the milk is instilled into the reservoir tank 4, passing through the drain pipe 6a and being ultimately collected in the secondary reservoir tank 6. The water containing the extracted ingredients from the blood or the milk which are collected in the secondary reservoir tank 6 is filtered in order to remove impurities, and subsequently, water containing extracted ingredients with effective ingredients from the blood or the milk as the primary ingredients is obtained as a final product.

Meanwhile, the atomized fine particles not liquefied in the condenser 3 pass through the communication pipe P4, are drawn in by the blower 5, and are circulated back to the atomized fine particle generating tank 1 together with the air flow; following this, they again pass through the plastic pipe P1 and are delivered to the extraction device 2.

As explained above, the effective ingredients of the blood constituting the raw material are captured into the atomized fine particles as a result of circulation thereof around the circulatory route, and an water containing extracted ingredients containing effective ingredients from the blood through liquefaction of the atomized fine particles; however, the duration of a single operation of the producing apparatus is one hour. That is to say, when extraction was carried out for one hour in accordance with the embodiment explained above and using approximately 800 grams of the absorber materials holding the raw materials, between approximately 2 and 3 liters of water containing extracted ingredients was ultimately produced.

Guide plates 2d were used within the inner cylinder 2c of the extraction device 2 in accordance with the embodiment explained above, and in contrast to non-usage thereof, this increased the collection volume of water containing extracted ingredients per unit time by approximately 20% and reduced the corresponding concentration.

Non-condensed atomized fine particles in the embodiment of the health drink producing apparatus as explained above are circulated back to the atomized fine particle generating tank 1 via the communication pipe P4 and the blower 5 as shown in FIG. 1, FIG. 2, and FIG. 3, and the temperature thereof drops to approximately 15° C. as a result of cooling in the cold storage chamber 7. In this cooled condition, furthermore, the non-condensed atomized fine particles mix with the atomized fine particles newly generated in the atomized fine particle generating tank 1 and are delivered to the plastic pipe 1; consequently, the temperature of the newly generated atomized fine particles drops, thus leading to condensation thereof and the formation of water drops, and the transfer of atomized fine particles in the plastic pipe P1 is thus obstructed. As a countermeasure for this problem, it is acceptable either to heat a portion of the communication pipe P4 disposed outside the cold storage chamber 7 or to rotate the atomized fine particles delivered to the interior of the atomized fine particle generating tank 1 from the blower 5 using a current plate formed so as to achieve a spiral shape, thus raising the temperature thereof over this interval before re-delivery thereof to the plastic pipe P1.

The water containing extracted ingredients obtained using the producing apparatus and producing method explained above is a transparent, colorless and clean liquid. The water containing extracted ingredients has a medical function and is effective as medicines for humans or animals. Specifically, these components are effective in terms of suppression of the occurrence and migration of cancer and tumors, and with respect to diseases such as leukemia, kidney disease, liver disease, hepatitis, diabetes, atopic dermatitis, high blood pressure, high cholesterol, arthritis, rheumatic arthritis, AIDS, brain damage, Alzheimer's disease, ear discharge, and Lyme disease.

Although blood or milk is used as the raw material for ingredient extraction in this embodiment of the present invention, this selection does not restrict the scope thereof. That is to say, known and unknown ingredients can be extracted from liquids or fluidity substances such as animal milk, starch syrup, wood acid, resin, sap, yogurt, cheese, miso soybean paste, soy sauce, soya milk, and fish sauce. In addition, plant, animal and mineral substances that are not normally liquids or fluidity substances can also be used as raw materials. Specifically, crushed or pulverized pieces of these substances mixed together with water can be used as the raw material.

Whereas extraction of ingredients from such substances is possible according to Japanese patent No. 3233187 and U.S. Pat. No. 5,558,006, the efficiency thereof is poor. Furthermore, the extraction method according to the present invention is capable of extracting ingredients that cannot be extracted by the technologies disclosed in those patents. For example, in the case of crystals, pearls, and tourmaline, a number of novel effective ingredients can be extracted when crushed or pulverized pieces thereof are mixed together with water and used as the raw material.

In accordance with the configuration and operation as explained above, the present invention is capable of the efficient low-temperature extraction of ingredients not extractable from various substances by conventional technologies.

Furthermore, water containing extracted ingredients produced from various substances can be used in many different ways in accordance with the desired application.

1. A method for producing water containing extracted ingredients extracted from plant, animal, and mineral matter, comprising the steps of;
(a) making a liquid or fluidity raw material come into contact with an absorber material and drying said absorber material to obtain a holding body of said raw material;
(b) generating atomized fine particles of water heated using a heater heating stored water to a predetermined temperature and an atomized fine particle generating tank providing a means for atomization of the water;
c) depressurizing a raw material layer comprising holding body according to the step (a) charged in an extraction device and sucking to and exuding out the surface of said atomized fine particles the effective ingredients in the raw materials;
(d) making the effective ingredients deposited on said raw material surface through suction and exudation hold onto the atomized fine particles by circulating said atomized fine particles together with a flow of air in a cyclinc fashion through the various component devices and passing thorugh the depressurized raw material layer;
(e) delivering the atomized fine particles holding the effective ingredients to a cooled condenser and condensing the particles;
(f) instilling into a reservoir tank the water containing the effective ingredients of the various raw materials after being condensed by the condenser, and obtaining the final product;
(g) re-circulating to the atomized fine particle generating tank the atomized fine particles not ocndensed in the condenser.

2. The method for producing water containing extracted ingredients of claim 1, wherein said absorber material is polyvinylidene fluoride.

3. The method for producing water containing extracted ingredients of claim 1, wherein said absorber material is glass fiber.

4. The method for producing water containing extracted ingredients of claim 1, wherein said absorber material is cellulose.

5. The method for producing water containing extracted ingredients of claim 1, wherein said raw material is a liquid or fluidity substance comprising crushed or pulverized plant, animal, or mineral matter with a necessary amount of water added thereto.

6. The method for producing water containing extracted ingredients of claim 1, wherein said liquid or fluidity raw material is blood, animal milk, wood acid, starch syrup, resin, sap, yogurt, cheese, fish sauce, miso soybean paste, soy sauce, soya milk, crystal-powder aqueous solution, tourmaline-powder aqueous solution, or pearl-powder aqueous solution.

7. The method for producing water containing extracted ingredients of claim 1, wherein the temperature of said water in said atomized fine particle generating tank is approximately 80° C. or less, and the temperature of said raw material layer and said atomized fine particles in said extraction device is between approximately 60° C. and 70° C.

8. Water containing extracted ingredients principally including ingredients extracted from plant, animal, and mineral matter and produced according to the following processes of;
(a) making a liquid or fluidity raw material come into contact with an absorber material and drying said absorber material to obtain a holding body of said raw material;
(b) generating atomized fine particles of water heated using a heater heating stored water to a predetermined temperature and an atomized fine particle generating tank providing a means for atomization of the water;
(c) depressurizing a raw material layer comprising holding body according to the process (a) charged in an extraction device and sucking to and exuding out the surface of said atomized fine particles the effective ingredients in the raw materials;
(d) making the effective ingredients deposited on said raw material surface through suction and exudation hold onto the atomized fine particles by circulating said atomized fine particles together with a flow of air in a cyclinc fashion through the various component devices and passing thorugh the depressurized raw material layer;
(e) delivering the atomized fine particles holding the effective ingredients to a cooled condenser and condensing the particles;
(f) instillating into a reservoir tank the water containing the effective ingredients of the various raw materials after being condensed by the condenser, and obtaining the final product;
(g) re-circulating to the atomized fine particle generating tank the atomized fine particles not condensed in the condenser.

9. The water containing extracted ingredients of claim 8, wherein said absorber material is polyvinylidene fluoride.

10. The water containing extracted ingredients of claim 8, wherein said absorber material is glass fiber.

11. The water containing extracted ingredients of claim 8, wherein said absorber material is cellulose.

12. The water containing extracted ingredients of claim 8, wherein said raw material is a liquid or fluidity substance comprising crushed or pulverized plant, animal, or mineral matter with a necessary amount of water added thereto.

13. The water containing extracted ingredients of claim 8, wherein said liquid or fluidity raw material is blood, animal milk, starch syrup, wood acid, resin, sap, yogurt, cheese, miso soybean paste, soy sauce, fish sauce, soya milk, crystal-powder aqueous solution, tourmaline-powder aqueous solution, or pearl-powder aqueous solution.

14. The water containing extracted ingredients of claim 8, wherein the temperature of said water in said atomized fine particle generating tank is approximately 80° C. or less, and the temperature of said raw material layer and said atomized fine particles in said extraction device is between approximately 60° C. and 70° C.
